(19) United States
(12) Patent Application Publication
(10) Pub. No.: US 2008/0014157 A1
(43) Pub. Date: Jan. 17, 2008

(54) Skin or hair treatment agents with protective effect

(75) Inventors: Jan Baumeister, Farvagny-le-Grand (CH); Paqual Dougoud, Marly (CH); Knut Meinert, Lampertheim (DE)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/800,825

(22) Filed: May 8, 2007

Related U.S. Application Data
(63) Continuation of application No. PCT/EP05/11990, filed on Nov. 9, 2005.

(30) Foreign Application Priority Data
Nov. 9, 2004 (DE) 102004054080.2

(51) Int. Cl. A61K 8/34; A61Q 17/04; A61Q 19/00; A61Q 5/00

(52) U.S. Cl. 424/59; 424/70.1

(57) ABSTRACT

The invention relates to agents for the protective, conditioning, detersive or cosmetic treatment of skin or hair that contain pyridinols or pyrimidinols or their mixtures.

### FIELD OF THE INVENTION

The invention relates to agents for the preventative, conditioning, detersive or cosmetic treatment of skin or hair that contain pyridinols or pyrimidinols or their mixtures.

### BACKGROUND OF THE INVENTION

The surface of the human body, especially the hair, is subject to numerous harmful effects. In addition to structural damage that is more hidden, these effects can also have more or less obvious visual actions.

For instance, even under normal, in no way excessive, exposure to sunlight, such severe hair damage can occur that visibly reduces the attractiveness of hair. Particularly when wet, hair can lose its luster and elasticity through the effect of light. It frequently becomes rough, brittle, and straw-like. However, the most visible impairment is usually hair damage that is associated with color change. In this way natural hair can discolor just like artificially applied oxidative hair colorants or direct-penetrating dyes.

As well as total loss of color, the hair can lose luster, depth, and brilliance. Since such changes do not usually occur uniformly the result is frequently an overall generally unattractive and patchy appearance of the hair. In the least favorable case, particularly with artificial hair colorants, there is no constant lightening, but a shift in the color tone owing to the different light sensitivities of the individual coloring components.

There is, therefore, the requirement for a product that can prevent the discoloring of hair by sunlight. Since the influence of high sunlight exposure is frequently associated with water, for example, a seaside vacation in the south, it is also desirable that the hair be provided with water-proof protection. Such protection must be retained, even when bathing in chlorinated or sea water. Furthermore, in this context a product that develops its hair-protecting action after exposure to sunlight would be desirable.

Numerous approaches for the suppression of light-induced color changes of human hair are known. Various attempts have been made with different levels of success to protect hair with UV absorbers that have been used very successfully in skin cosmetics.

Despite the ostensible logic of such approaches to the problem they usually fail because only very small layers can be applied to hair without objectionable effects in appearance becoming obvious. Thus gray hair frequently appears yellow with the use of common UV absorbers owing to their intrinsic color; on the other hand light-scattering pigments based on titanium dioxide are objectionally visible, especially on dark hair.

Moreover, it is not a simple matter to produce a coherent protective film without adverse effects such as oily appearance and sticky feel to the hair. In addition, unlike the formation of sunburn on the skin, hair color is not only damaged by UV light but also by the whole frequency range of the visible spectrum. In order to screen visible light, dark, or high light-scattering (thus in themselves color-modifying) protectants must be applied to the hair.

On the supposition that bleaching processes are oxidative or radical-induced processes the use of reducing agents and radical scavengers have been varyingly attempted. In isolated cases measurable photoprotective actions have been detected with some natural or synthetic antioxidants; however, a clearly visible effect could thus far not be achieved.

It is neither possible nor desirable to protect the hair consistently from the cited effects since being outdoors contributes to the quality of life, just like cleaning and styling the hair. Besides, no method of hair styling or color change is known that develops its action without leaving a certain amount of damage. Since hair is not subject to any biological regeneration process after its formation it is subject to substantial damage caused by external influences the prejudicial effects of which progress further with increasing lifetime of the hair.

The properties of pyrimidinol and pyridinol derivatives as antioxidants that interrupt radical reactions are known. In particular, the use of such compounds as fuel additives, lubricant additives, polymer additives, solvent additives and foodstuffs additives has been described. In addition, the suitability of the compounds as components of medication for the slowing of aging or prevention of cancer, cardiac or pulmonary diseases, inflammation, Alzheimer's or Parkinson's disease has been described. Moreover, the compounds can be used as oxidation inhibitors in cosmetic products as hair liquid, cream, and emulsions. However, the suitability of the described compounds as components of cosmetic compositions for the protection of hair and skin towards sun radiation and chemical agents as well as for the conditioning and cleaning of hair and skin, in particular as active agent in cosmetic hair compositions, is not currently known.

Pratt et al. describe in “Chem. Eur. J. 2003, 9, 4997-5010” the effect of a nitrogen atom in the ring in relation to the homolytic reactivity of phenol compounds. The ability of 5-pyrimidinols to trap radicals is particularly investigated. According to the publication, six substituted pyrimidinols were prepared and their thermochemistry and kinetics investigated in respect of their reaction with free radicals. The compounds were described as antioxidants and potential components of anti-inflammatory and cytoprotective medicaments. The use of corresponding compounds for the preventative, conditioning, detersive or cosmetic treatment of skin or hair has not been described in the publication.

European Patent No. 0 210 044 A2 relates to hydroxy and alkoxypyrimidines. 2-Amino-4-substituted-5-(hydroxy or alkoxy)pyrimidines that can be substituted in the 6 position are especially described. The compounds are termed inhibitors of leukotriene synthesis and are accordingly considered suitable for the treatment of inflammation, allergic reactions, cardiovascular diseases, or respiratory diseases. Use of the compounds named in agents for preventative, conditioning, detersive or cosmetic treatment of skin or hair is not described in the publication.

U.S. Pat. No. 5 196 431 A describes 2-substituted amino-4,6-di-t-butyl-5-hydroxy-1,3-pyrimidines and pharmaceutically acceptable salts thereof, pharmaceutical compositions, and methods of use therefor. The compounds are having activity as inhibitors of 5-lipoxygenase and cyclooxygenase providing treatment of medical conditions such as inflammation, arthritis, pain, fever, and the like. In addition, the compounds can be used topically for therapeutical treatment of medical conditions as acne, sunburn, psoriasis, and eczema.

European Patent No. 0 373 827 B1 relates to the use of certain derivatives of 5-hydroxy- and 5-methoxy-2 aminopyrimidines and the pharmaceutically-acceptable salts thereof to inhibit interleukin-1 production in a mammal. The compounds are used for the therapeutical treatment of interleukin-1 mediated disorders and dysfunctions such as osteoporosis, allergies, and psoriasis, whereas the compounds are desirably administered topically in doses ranging from 0.1 mg/kg up to 100 mg/kg body weight, e.g., in lotions, creams, and gels.

European Patent No. 0 138 464 B1 describes 2-amino-5-hydroxy-4-methylpyrimidines, their pharmaceutically-acceptable salts and the preparation thereof as well as therapeutical compositions comprising those compounds in a pharmaceutically-effective amount. The compounds are valuable agents in the treatment of asthma, inflammation, cardiovascular spasm, psoriasis, and cancer. Moreover, the compounds have “cytoprotective” properties, for example, they inhibit ulcer formation in rats. The compounds can be administered topically in a therapeutically-effective dose ranging from 1 mg/kg to 100 mg/kg, in particular from 1 mg/kg to 20 mg/kg, body weight per day.

“Angew. Chem. Int. Ed., 2003, 42, 4370-4373” relates to the calculation and experimental determination of O—H bond enthalpies in 3-pyridinols und 5-pyrimidinols as well as the synthesis of some new 3-pyridinols. It is pointed out that the O—H bond enthalpies play a central role in determining antioxidant efficacy of those compounds. 6-Amino-3-pyridinols are described as being more effective antioxidants than any other antioxidants of the phenolic class.

German Patent No. 600 07 046 T2 relates to a cosmetic composition containing at least one silicone-acrylate copolymer and at least one photoprotective agent. Cinnamic acid derivatives are used as photoprotective agents, for example, isopentyl 4-methoxycinnamate, as well as pigments or nanopigments with a mean size of the primary particles of 5 nm to 100 nm.

Common to the known solutions, is that they frequently exhibit the disadvantages already described above regarding their behavior on the skin or hair in respect of their distribution and in respect of their protective action. Thus, there was a requirement for compounds that exhibit an antioxidative action in cosmetic preparations and hence can be used, for example, as sun screens or UV protection for hair and skin.

The task that is the basis of the present invention is therefore to make available a composition that avoids the disadvantages of the known compositions. Furthermore, the fundamental task of the present invention is to make available a composition for the treatment of the surface of the body of a living being that protects the body surface from external environmental effects, especially high-energy radiation, for example, UV radiation, or the consequences of such radiation whereby in the most part the external appearance of the surface of the body, for example the hair, is not unfavorably affected any more than is avoidable.

It was found that unlike the normal commercial UV absorbers, known natural or synthetic reducing agents, antioxidants or also radical scavengers such as ascorbic acid (vitamin C), tocopherol (vitamin E) or butylhydroxyanisole (trade name Oxynex), suitable pyrimidinols and pyridinols can exert an advantageous protection against UV-related damage to hair even in small layers. It emerged that the pyrimidinols and pyridinols can fulfill the demands that are placed on such compounds in respect of their use in agents for the preventative, conditioning, detersive or cosmetic treatment of skin or hair. It was especially established that such agents that contain certain pyrimidinols or pyridinols or their mixture protect hair structure excellently from UV-related hair damage.

### DETAILED DESCRIPTION

The present invention relates to a compound of the general formula I 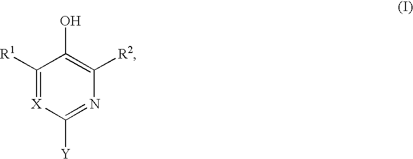
wherein X stands for N or C, R¹ for a linear or branched, saturated or unsaturated C₁₋₂₄-alkyl residue, a phenyl residue, a C₇₋₂₈-phenylalkyl residue, whereby the phenylalkyl residue can be substituted on the phenyl ring by fluorine, chlorine, C₁₋₆-alkyl, C₁₋₆-alkoxy. CF₃, furfuryl or thienyl, R² stands for hydrogen, C₁₋₂₄-alkyl, phenyl, C₇₋₂₈-phenylalkyl, whereby the phenylalkyl on the phenyl ring can be substituted by fluorine, chlorine, C₁₋₆-alkyl, C₁₋₆-alkoxy, CF₃, furfuryl or thienyl, and Y stands for NH₂, NHR³ or NR³R⁴, wherein R³ and R⁴, independently of one another, stand for a linear or branched, saturated or unsaturated C₁₋₁₂-alkyl group or a linear or branched, saturated or unsaturated C₁₋₁₂-hydroxyalkyl group, or R³ and R⁴ together form a ring so that Y stands for an imidazolyl group or a quinolinyl group, or a morpholinyl group or a pyrazolyl group or a piperidinyl group or Y stands for a linear or branched, saturated or unsaturated C₁₋₁₂-alkoxy group, a linear or branched, saturated or unsaturated C₁₋₁₂-hydroxyalkoxy group, a linear or branched, saturated or unsaturated C₁₋₁₂-alkylcarboxylic acid group, a linear or branched, saturated or unsaturated C₁₋₂₄-alkylcarboxamide group, a linear or branched, saturated or unsaturated C₁₋₁₂-alkylsufonic acid group, a linear or branched, saturated or unsaturated C₁₋₂₄-alkylsulfonic acid ester group or a linear or branched, saturated or unsaturated C₁₋₂₄-alkylsulfonamide group or wherein R³ together with X forms an anelated cyclic system of 5 or 6 atoms, or a physiologically compatible salt of such a compound of general formula I or an isomeric or stereoisomeric form of a compound of general formula I, as component of an agent for the preventative, conditioning, detersive or cosmetic treatment of skin or hair.

Suitable physiologically compatible salts of the general compounds of general formula I are, for example, alkali, alkaline earth, ammonium, triethylamine or tris(2-hydroxyethyl)amine salts as well as those that are formed from the transformation of compounds of formula I with inorganic or organic acids such as hydrochloric acid, phosphoric acid, sulfuric acid, branched or unbranched substituted (for example, by one or more hydroxy groups) or unsubstituted C₁₋₄-mono- or dicarboxylic acids, aromatic carboxylic acids and sulfonic acids such as acetic acid, citric acid, benzoic acid, maleic acid, fumaric acid, tartaric acid, and p-toluenesulfonic acid. Examples of suitable physiologically compatible salts are the Na, K, Mg, Ca, and ammonium salts of compounds according to formula I as well as the salts that are formed by transformation of compounds according to structure (I) or (II) with hydrochloric acid, acetic acid, citric acid, and benzoic acid.

Isomeric and stereoisomeric forms of the compounds of the general formula I according to the invention are understood to be all their occurring optical isomers, diastereomers, racemates, zwitterions, cations, or mixtures thereof.

In many cases it has been established as advantageous if within the context of the use of the invention a compound of the general formula I is used where R¹ and R², independently of one another, stand for a linear or branched C₁₋₁₂-alkyl residue or C₁₋₆-alkyl residue, a phenyl residue or a phenyl residue substituted on the phenyl ring with C₁₋₆-alkyl or C₁₋₆-alkoxy.

In regard to the nature of the substituent Y, within the context of the present invention compounds can be used, for example, in which Y stands for NH₂, NHR³, or NR³R⁴, whereby R³ and R⁴, independent of one another, stand for a linear or branched C₁₋₆-alkyl group or a linear or branched saturated or unsaturated C₁₋₆-hydroxyalkyl group, or Y stands for a linear or branched C₁₋₆-alkoxy group, a linear or branched C₁₋₆-hydroxyalkyoxy group, a linear or branched C₁₋₆-alkylcarboxylic acid group, a linear or branched C₁₋₆-alkylcarbamoyl group, a linear or branched C₁₋₁₂-C₁₋₆-alkylsulfonic acid group, a linear or branched C₁₋₁₂-alkylsulfonic acid ester group or a linear or branched C₁₋₁₂-alkylsulfonamide group.

Within the context of a further embodiment of the present invention it has been shown to be advantageous when R¹ and R², independently of one another, stand for a methyl or ethyl residue. Especially suitable compounds of the general formula I are, for example, 2-dimethylamino-4,6-dimethyl-5-pyrimidinol, 2-diethylamino-4,6-dimethyl-5-pyrimidinol, 2-dimethylamino-4,6-diethyl-5-pyrimidinol or 2-diethylamino-4,6-diethyl-5-pyrimidinol or 2-methylethylamino-4,6-dimethyl-5-pyrimidinol or 2-methylethylamino-4,6-diethyl-5-pyrimidinol and the like.

Within the context of the present invention compounds of the general formula I can in each case be used individually or as mixture of two or more thereof. The method according to the invention includes the method of using individual pyridinols or pyrimidinols or the method of using a mixture of two or more pyridinols or the method of using a mixture of two or more pyrimidinols or the method of using a mixture of a pyridinol and a pyrimidinol or the method of using a mixture of two or more pyridinols and a pyrimidinol or the method of using two or more pyrimidinols with a pyridinol or the method of using a mixture of two or more pyridinols and a mixture of two or more pyrimidinols.

The compounds of the general formula I can be used together with all materials customary within the scope of the above uses in products for skin and hair treatment. The active compounds can be present dissolved, emulsified (w/o and o/w), suspended, in liquid crystalline phase, as aerosol, gel, wax, powder, or foam. Compounds of the general formula I can be used as highly effective protectants even in a concentration range of about 0.00001% to about 20%; preferably between about 0.001% and about 5%, for example, between about 0.0001% to about 2%; more preferably between about 0.01% and about 1%, for example, between about 0.01% and about 0.5%, for example, between about 0.01% and about 0.1% for the protection of hair from damage by sun radiation or chemical agents. Naturally a compound of the general formula I or a mixture of two or more such compounds can be used alone or also with other antioxidants/radical scavengers. The agents concerned are applied before, during, or after sunbathing in the open or in a solarium or a chemically aggressive hair treatment. The pH value of the product concerned can lie between 2.0 as, for example, in an acid regime, or 14.0 (alkali relaxer). The pH lies preferably between 3.0 and 9.0. The respective agents are described more closely in the following.

In respect of the amount of pyridinols or pyrimidinols or their mixtures as discussed in detail above it has been demonstrated that within the scope of a broad concentration range a corresponding activity spectrum exists. The method according to the invention thus includes the use of the compounds of the general formula I or a mixture of two or more such compounds in an amount of from about 0.0001% by weight to about 15% by weight or from about 0.0005% by weight to about 10% by weight. Suitable activity ranges are found, for example, within a quantity range from about 0.001% by weight to about 5% by weight or from about 0.005% by weight to about 3% by weight, or from about 0.01% by weight to about 2% by weight, or from about 0.1% by weight to about 1.5% by weight.

A method to the present invention categorically encompasses every method of using of a compound of the general formula I in an agent that serves the preventative, conditioning, detersive or cosmetic treatment of skin or the preventative, conditioning, detersive or cosmetic treatment of hair. In principle, thereby, the treatment of inflammatory medical indications is in principle not included if a compound of the general formula I is a definitive part of such a treatment. According to the present invention by skin or hair is meant the skin or hair of mammals, in particular human skin or human hair

By “preventative treatment of skin or hair,” is meant treatment with which the skin or the hair is treated with an agent of the invention in order to prevent future damage, in particular future damage caused by environmental influences, for example, damage caused by UV radiation. By conditioning treatment of skin and hair is meant a treatment by which the skin or the hair is treated with an agent that contains a compound of the general formula I whereby the agent is suitable to improve the skin or the hair in respect of mechanical or haptic properties, for example, the elasticity of the skin or the hair, or the compatibility or tensile strength of the hair. By detersive treatment of skin and hair is meant a treatment with which the skin or the hair is treated with an agent that contains a compound of the general formula I whereby the agent is suitable to remove contaminants from the skin or hair. By cosmetic treatment of the skin or the hair is meant every treatment of skin or hair that leads to a change in the external appearance of the skin or hair. It is emphasized in this connection that a demarcation of respective agents from each other in respect of their action is often difficult. Thus also included within the scope of the present invention are those applications of the invention with which an agent that contains a compound of the general formula I exhibits two or more of the aforementioned actions, for example, a detersive and a cosmetic action, or a detersive, conditioning and cosmetic action, or further appropriate combinations.

Within the scope of a preferred embodiment of the present invention the use according to the invention relates to the use of a compound of the general formula I or a mixture of two or more such compounds in a conditioner, deep conditioner, deep conditioner cream, leave-on spray deep conditioner, hair treatment agent with volatile materials, hair styling gel, hair styling foam, hair spray, spray gloss product, styling pomade, permanent wave agent, agent for after-care after wave or color treatment, hair wax, shampoo, shower cream, sun screen, or hair & body sun screen spray.

The agents described according to the invention can contain all active materials, additives and adjuvants known for such preparations. These are, for example, gelling agents and/or thickeners, anionic polymers, surfactants, hydrating agents, emollients, hydrophilic or lipophilic active compounds such as ceramides, agents against free radicals, sequestriants, antioxidants, preservatives, alkalizers or acidifiers, perfumes, fillers, colorants, volatile or non-volatile, modified or non-modified silicones, and reducing agents.

The proportion of different additives are those that are used in the respective areas. The person skilled in the art will naturally so select the optionally present compound(s) incorporated into the composition according to the invention that the advantageous properties associated with the composition according to the invention are not or not significantly modified by the foreseen addition.

In many cases the agents contain at least one surfactant, in which case anionic as well as zwitterionic, ampholytic, non-ionic and cationic surfactants are suitable in principle. However, in many cases it has proved to be advantageous to select the surfactants from anionic, cationic, zwitterionic or non-ionic surfactants.

All anionic surface-active substances suitable for use on the human body are suitable as anionic surfactants in the preparations according to the invention. These are characterized by a water solubility-mediating anionic group, for example, a carboxylate, sulfate, sulfonate or phosphate group and a lipophilic alkyl group with about 10 C atoms to about 22 C atoms. In addition, glycol or polyglycol ethers, esters, ethers and amino groups as well as hydroxy groups may be present in the molecule. Examples of suitable anionic surfactants, in each case in the form of sodium, potassium and ammonium as well as mono-, di- and trialkanolammonium salts with about 2 C atoms or about 3 C atoms in the alkanol group, are
linear fatty acids with 10 C atoms to 22 C atoms (soaps),
carboxylic acid ethers of the structure R—O—(CH₂—CH₂O)X—CH₂—COOH in which R stands for a linear alkyl group with 10 C atoms to 22 C atoms and X stands for 0 or 1 to 16,
acyl sarcosides with 10 C atoms to 18 C atoms in the acyl group,
acyl taurides with 10 C atoms to 18 C atoms in the acyl group,
acyl isethionates with 10 C atoms to 18 C atoms in the acyl group,
sulfonsuccinic acid mono- and dialkyl esters with 8 C atoms to 18 C atoms in the alkyl group and sulfonsuccinic acid monoalkylpolyoxyethyl esters with 8 C atoms to 18 ⁻C atoms in the alkyl group and 1 to 6 oxyethyl groups,
linear alkane sulfonates with 12 C atoms to 18 C atoms,
linear alpha-alkene sulfonates with 12 C atoms to 18 C atoms;
methyl alpha-sulfonates of fatty acids with 12 C atoms to 18 C atoms,
alkyl sulfates and alkyl polyglycol ether sulfates of the structure R—O(CH₂—CH₂O)ₓ—SO3H in which R stands preferably for a linear alkyl group with 10 C atoms to 18 C atoms and x stands for 0 or 1 to 12,
mixtures of surface-active hydroxysulfonates as described in German Patent No. 37 25 030,
sulfated hydroxyalkylpolyethylene and/or hydroxyalkylenepropyleneglycol ethers as described in German Patent No. 37 23 354,
sulfonate of unsaturated fatty acids with 12 C atoms to 24 C atoms and 1 to 6 double bonds as described German Patent No. 39 26 344,
esters of tartaric acid and citric acid with alcohols that represent addition products of about 2 to 15 molecules ethylene oxide and/or propylene oxide to fatty alcohols with 8 C atoms to 22 C atoms.

Preferred anionic surfactants are alkyl sulfates, alkylpolyglycol ether sulfates and ether carboxylic acids with 10 C atoms to 18 C atoms in the alkyl group and up to 12 glycol ether groups in the molecule as well as especially salts of saturated or unsaturated C₈-C₂₂ carboxylic acids such as oleic acid, stearic acid, isostearic acid, and palmitic acid.

Non-iogenic surfactants contain as hydrophilic group, for example, a polyol group, a polyalkyleneglycol ether group or a combination of polyol and polyglycol ether groups. Such compounds are, for example,
addition products of 2 mol to 30 mol ethylene oxide and/or 0 mol to 5 mol propylene oxide to linear fatty acids with 8 C atoms to 22 C atoms, to fatty acids with 12 C atoms to 22 C atoms and to alkylphenols with 8 C atoms to 15 C atoms in the alkyl group,
C₁₂-C₂₂ fatty acid mono⁻ and diesters of addition products of 1 mol to 30 mol ethylene oxide to glycerol,
C₈-C₂₂-alkylmono- and oligoglycosides and their ethoxylated analogues as well as addition products of 5 mol to 60 mol ethylene oxide to castor oil and hydrogenated castor oil.

Suitable non-ionic surfactants are furthermore alkylpolyglycosides of the general formula RO-(Z)ₓ, wherein the alkyl residue R has 6 C atoms to 22 C atoms and can be both linear and branched. Preferred are primary linear aliphatic residues and those branched with methyl at the 2 position. Such alkyl residues are, for example, 1-octyl, 1-decyl, 1-lauryl, 11myristyl, 1-cetyl and 1-stearyl. Especially preferred are 1-octyl, 1-decyl, 1-lauryl, imyristyl. In the use of so-called “oxo alcohols” as starting materials compounds with an odd number of carbon atom in the alkyl claim predominate.

The alkyl polyglycosides utilizable according to the invention can contain, for example, only one definitive alkyl residue R. Normally, however, these compounds are prepared starting from natural fats and oils or mineral oils. In this case mixtures corresponding to the starting compounds or to the respective work-up of these compounds are present as the alkyl residue R.

Particularly preferred are those alkylpolyglycosides in which R consists substantially of C₈ and C₁₀ alkyl groups,
substantially of C₁₂- and C₁₄-alkyl groups,
substantially of C₈-C₁₆-alkyl groups or
substantially of C₁₂-C₁₆-alkyl groups.

The alkylpolyglycosides can have any desired mono- or oligosaccharide as sugar building block Z. They normally contain sugars with 5 C atoms or 6 C atoms or the corresponding oligosaccharides. Such sugars are, for example, glucose, fructose, galactose, arabinose, ribose, xylose, lyxose, allose, altrose, mannose, gulose, idose, talose, and sucrose. Preferred sugar building blocks are glucose, fructose, galactose, arabinose, and sucrose; glucose is particularly preferred.

The alkyl polyglycosides utilizable according to the invention contain on average 1.1 sugar units to 5 sugar units. Alkylpolyglycosides with x values of 1.1 sugar units to 1.6 sugar units are preferred. Alkylglycosides where x is 1.1 sugar units to 1.4 sugar units are also suitable.

The alkoxylated homologues of the named alkyl polyglycosides can also be used according to the invention. These homologues can contain on average up to 10 ethylene oxide and/or propylene oxide units per alkylglycoside unit.

Furthermore, zwitterionic surfactants can be used, especially as co-surfactants. Zwitterionic surfactants are those surface-active compounds that bear in the molecule at least one quaternary ammonium group and at least one —COO⁻— or —SO₃⁻ group. Especially suitable zwitterionic surfactants are the so-called betaines, for example, the N-alkyl-N,N-dimethyl ammonium glycinates, for example, the cocoalkyldimethylammonium glycinate, N-acylaminopropyl-N,N-dimethylammonium glycinates, for example, the cocoacylaminopropyldimethylammonium glycinate, and 2-alkyl-3-carboxymethyl-3-hydroxyethylimidazolines with in each case 8 C atoms to 18 C atoms in the alkyl or acyl group as well as the cocoacylaminoethylhydroxyethylcarboxymethyl glycinate. A preferred zwitterionic surfactant is the fatty acid amide derivative known under the INCI designation cocamidopropyl betaine.

Equally especially suitable as co-surfactants are ampholytic surfactants. Ampholytic surfactants are understood to be those surfactants that in addition to C₈-C₁₈-alkyl or acyl groups in the molecule contain at least one free amine group and at least one —COOH— or —SO₃H group and are capable of forming internal salts. Examples of suitable ampholytic surfactants are N-alkylglycines, N-alkylpropanoic acids, N-alkylaminobutanoic acids, N-alkyliminodipropanoic acids, N-hydroxyethyl-N-alkylamidopropylglycines, N-alkyltaurines, N-alkylsarcosines, 2-alkylaminopropanoic acids and alkylaminoethanoic acids with in each case about 8 C atoms to 18 C atoms in the alkyl groups. Especially preferred ampholytic surfactants are N-cocosalkylaminopropionate, cocosacylaminoethylaminopropionate and C₁₂₋₁₈ acylsarcosine.

According to the invention quaternary ammonium compounds, ester quats, and amidoamines are especially used as cationic surfactants.

Preferred quaternary ammonium compounds are ammonium halides, especially chlorides and bromides such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known by the INCI notations Quaternium27 and Quaternium-83. The long alkyl chains of the above-mentioned surfactants have preferably 10 C atoms to 18 C atoms.

Ester quats are known compounds that contain both at least one ester function and at least one quaternary ammonium group as structural element. Preferred ester quats are quaternary ester salts of fatty acids with triethanolamine, quaternary ester salts of fatty acids with diethanolalkylamines and quaternary ester salts of fatty acids with 1,2-dihydroxypropyldialkylamines. Such products are marketed, for example, under the trade marks Stepantex®, Dehyquart®, and Armocare®. The products Arnlocare® VGH-70, a N,N-bis(2-palmitoyloxyethyl)dimethylammonium chloride, as well as DehyquarC F-75 and DehyquarC AU-35 are examples of such ester quats.

The alkylamidoamines are usually prepared by amidation of natural or synthetic fatty acids and fatty acid fragments with dialkylaminoamines. A particularly suitable compound of this substance group according to the invention is represented by stearamidopropyldimethylamine, commercially available under the name Tegoamid® S 18.

Further cationic surfactants that can be used according to the invention are the quaternized protein hydrolysates. According to the invention equally suitable are cationic silicone oils such as, for example, the commercially available products Q2-7224 (manufacturer: Dow Corning; a stabilized trimethylsilylamodimethicone), Dow Corning 929 emulsion (containing a hydroxylamino-modified silicone, also known as Amodimethicone), SM-2059 (manufacturer: General Electric), SLM-55067 (manufacturer: Wacker) as well as Abil®-′ Quat 3270 and 3272 (manufacturer: Th. Goldschmidt; diquaternary polydimethylsiloxane, Quaternium-80).

An example of a quaternary sugar derivative that may be used as a cationic surfactant is the commercially available Glucquat©100, according to the INCI nomenclature a “lauryl methyl gluceth-10 hydroxypropyldimonium chloride.”

In the case of the compounds with alkyl groups usable as surfactants they can be homogeneous substances. However, it is usually preferred to assume that in the production of these materials from natural vegetable or animal raw materials substance mixtures with different alkyl chain lengths are obtained dependent upon the respective raw material.

In the case of surfactants that are addition products of ethylene and/or propylene oxide to fatty alcohols or derivatives of these addition products both products with a “normal” homologue distribution and those with a narrow homologue concentration can be used. “Normal” homologue distribution is understood to mean mixtures of homologues that are obtained by the reaction of fatty alcohols with alkylene oxides in the presence of alkali metals, alkali metal hydroxides or alkali metal alcoholates as catalysts. Narrow homologue distributions are obtained, for example, when hydrotalcite, alkaline metal salts of ether carboxylic acids, alkaline earth metal oxides, hydroxides or alcoholate are used as catalysts. The use of products with narrow homologue distribution may be preferred.

Furthermore, the agents according to the invention can preferably contain a conditioning agent selected from the group consisting of cationic surfactants, cationic polymers, alkylamidoamine, paraffin oils, vegetable oils, and synthetic oils.

Preferred conditioning agents can be cationic polymers. These are usually polymers that contain a quaternary nitrogen atom, for example, in the form of an ammonium group.

Preferred Cationic Polymers are, for Example:

quaternized cellulose derivates, such as are commercially available under the names Celquat© and Polymer JR®. The compounds Celqua ® H 100, Celquat® L 200- and Polymer JR®400 are preferred quaternized cellulose derivates;
polymeric dimethyldiallylammonium salts and their copolymers with acrylic acid as well as esters and amides of acrylic acid and methacrylic acid. The products commercially available under the names Merquat©100 (poly(dimethyldiallylammonium chloride)), Merquat©550 (dimethyldiallylammonium chloride-acrylamide copolymer), and Merquat® 280 (dimethyldiallylammonium chloride-acrylic acid copolymer) are examples of such cationic polymers;
copolymers of vinylpyrrolidone with quaternary derivates of dialkylaminoacrylate and methacrylate, for example, with diethylsulfate-quaternised vinylpyrrolidone-dimethylaminomethacrylate copolymers. Such compounds are available commercially under the names Gafquat®734 and Gafquat©755.
vinylpyrrolidone-methoimidazolinium chloride copolymers that are marketed under the name Luviquat®;
quaternized polyvinyl alcohol as well as polymers with quaternary nitrogen atoms in the main polymer chain that are known as Polyquaternium 2, Polyquaternium 17, Polyquaternium 18, and Polyquaternium 27.

Particularly preferred are cationic polymers of the four first named groups, quite especially preferred are Polyquaternium-2, Polyquaternium-10, and Polyquaternium-22.

Also suitable as conditioning agents are silicone oils, especially dialkyl- and alkylarylsiloxanes, for example, dimethylpolysiloxane and methylphenylpolysiloxanes, as well as their alkoxylate and quaternized analogues. Examples of such silicones are the products marketed by Dow Corning under the names DC 190, DC 200, DC 344, DC 345, and DC 1401 as well as the trade products Q2-7224 (manufacturer: Dow Corning; a stabilized trimethylsilylamodimethicone), Dow Corning 929 Emulsion (containing a hydroxyamino-modified silicone, that is also called Amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxane, Quaternium-80).

Equally usable as conditioning agents are paraffin oils, synthetic oligomeric alkenes as well as vegetable oils such as jojoba oil, sunflower oil, orange oil, almond oil, wheat germ oil, and peach stone oil.

Likewise suitable hair conditioning compounds are phospholipids, for example, soy lecithin, egg lecithin, and cephalines.

Further active compounds, adjuvants and additives are, for example:
nonionic polymers such as vinylpyrrolidone/vinylacrylate copolymers, polyvinylpyrrolidone and vinylpyrrolidone/vinyl acetate copolymers, and polysiloxanes;
zwitterionic and amphoteric polymers such as acrylamidopropyltrimethylammonium chloride/acrylate copolymers and octylacrylamide/methylmethacrylate/tert-butylaminoethylmethacrylate/2-hydroxypropylmethacrylate copolymers;
anionic polymers, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methylvinyl ether/maleic acid anhydride copolymers, and acryl acid/ethyl acrylate/N-tert.butylacrylamide terpolymers;
thickening agents such as agar, guar gum, alginate, xanthan gum, gum arabic, karaya gum, carob bean flour, linseed gum, dextrans, cellulose derivatives, e.g., methylcellulose, hydroxyalkylcellulose and carboxymethylcellulose, starch fractions and derivatives such as amylose, amylopectin and dextrins, clays such as bentonite or fully synthetic hydrocolloids such as polyvinyl alcohol;
structuring agents such as maleic acid and lactic acid;
hair conditioning compounds such as phospholipids, for example, soy lecithin; egg lecitin and cephalines, protein hydrolysates, especially elastin, collagen, keratin, milk protein, soy protein and wheat protein hydrolysates, their condensation products with fatty acids as well as quaternized protein hydrolysates;
perfume oils, dimethylisosorbide, and cyclodextrins;
solvents and solubilizers such as ethanol, isopropanol, ethylene glycol, propylene glycol, glycerol, and diethylene glycol;
fiber structure improvement agents, especially mono-,di- and oligosaccharides such as glucose, galactose, fructose, D-fructose, and lactose;
quaternary amines such as methyl-1-alkylamidoethyl-2-alkylimidazolinium methosulfate;
foam inhibitors such as silicones, dyes for coloring the agent;
active antidandruff agents such as piroctone olamine, zinc pyrithione, and climbazole;
photoprotective agents, especially derivatized benzophenones, cinnamic acid derivatives, and triazines;
buffers, for example normal acids, especially benefit acids and bases;
plant extracts such as the extracts from green tea, oak bark, stinging nettles, witch hazel, hops, chamomile, burdock root, horsetail, hawthorn, lime blossom, almonds, aloe vera, pine needles, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, marshmallow, meristem, ginseng, and ginger root;
cholesterol;
consistency regulators such as sugar esters, polyol esters or polyol alkyl ethers, fats and waxes such as spermaceti, beeswax, montanin wax and paraffins, fatty acid alkanolamides;
chelating agents such as EDTA, NTA, β-alanine diacetic acid and phosphoric acids;
swelling and penetration agents such as glycerol, propylene glycol monoethyl ether, carbonates, hydrogen carbonates, guanidines, ureas as well as primary, secondary, and tertiary phosphates;
opacifiers such as latex, styrene/PVP, and styrene/acrylamide copolymers;
pearlescents such as ethyleneglycol mono- and distearate as well as PEG-3 distearate, pigments;
stabilizers for hydrogen peroxide and other oxidizing agents;
aerosol propellants such as propane-butane mixtures, N20, dimethyl ether, C02, and air;
antioxidants.

In respect of other facultative components as well as the amounts of compounds used expressed reference is made to the comprehensive reference books such as “Kh. Schrader, Grundlagen und Rezepturen der Kosmetika, 2. edition, H\ddot{ut}hig Buch Verlag, Heidelberg, 1989.”

Within the context of the present invention it is evident that the use of the compounds of the general formula I in an agent for the protective, conditioning, detersive or cosmetic treatment of skin or hair in many cases brings about a significant improvement in properties compared with the known agents. In this connection it is necessary that a pertinent agent contains at least one compound of the general formula I, at least one liquid carrier, and at least one surfactant.

Object of the present invention is thus an agent for the protective, conditioning, detersive or cosmetic treatment of skin or hair containing at least one liquid carrier, one surfactant, and one compound of the general formula I 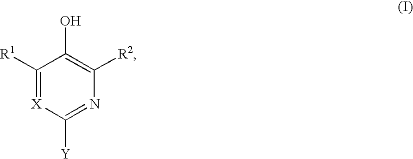
wherein X stands for N or C, R¹ for a linear or branched, saturated or unsaturated C₁₋₂₄-alkyl residue, a phenyl residue, a C₇₋₂₈-phenylalkyl residue, whereby the phenylalkyl residue can be substituted on the phenyl ring by fluorine, chlorine, C₁₋₆-alkyl, C₁₋₆-alkoxy. CF₃, furfuryl or thienyl, R² stands for hydrogen, C₁₋₂₄-alkyl, phenyl, C₇₋₂₈-phenylalkyl, whereby the phenylalkyl can be substituted on the phenyl ring with fluorine, chlorine, C₁₋₆-alkyl, C₁₋₆-alkoxy. CF₃, furfuryl or thienyl, and Y stands for NH₂, NHR³ or NR³R⁴, wherein R³ und R⁴, independently of one another, stands for a linear or branched, saturated or unsaturated C₁₋₁₂-alkyl group or a linear or branched, saturated or unsaturated C₁₋₁₂-hydroxyalkyl group, or R³ and R⁴ together form a cyclic system so that Y stands for a imidazolyl group or a quinolinyl group, or a morpholinyl group or a pyrazolyl group or a piperidinyl group or Y stands for a linear or branched, saturated or unsaturated C₁₋₁₂-alkoxy group, a linear or branched, saturated or unsaturated C₁₋₁₂-hydroxyalkoxy group, a linear or branched, saturated or unsaturated C₁₋₁₂-alkylcarboxylic acid group, a linear or branched, saturated or unsaturated C₁₋₂₄-alkylcarbamoyl group, a linear or branched, saturated or unsaturated C₁₋₁₂-alkylsulfonic acid group, a linear or branched, saturated or unsaturated C₁₋₂₄-alkylsulfonic acid ester group or a linear or branched, saturated or unsaturated C₁₋₂₄-alkylsulfonamide group or wherein R³ together with X forms an anelated cyclic system of 5 atoms or 6 atoms, or a physiologically compatible salt of such a system of the general formula I or an isomeric or stereoisomeric form of a compound of general formula I, or a mixture of two or more thereof, whereby the content of the agent of compounds of the general formula I is less than 10%.

All skin or hair compatible liquids that as carrier substances allow on the one hand an effective distribution of a respective agent and on the other hand correspondingly allow and preferably support the application of such content materials onto skin or hair are suitable as liquid carriers.

A suitable liquid carrier contains especially water and optionally cosmetically acceptable organic solvents. Suitable liquid organic carriers are, for example, compounds selected from the group of organic solvents, for example alcohols, esters, ketones, paraffins, or ester alcohols.

The organic solvents can consist of about 0% by weight to about 99% by weight, for example, 0.5% by weight to about 90% by weight of the total weight of the liquid carrier substance. They can be selected from hydrophilic organic solvents, lipophilic organic solvents, amphiphilic solvents, or their mixtures.

Of the hydrophilic organic solvents the straight-chain or branched lower monoalcohols with 1 C atom to 8 C atoms, the polyethylene glycols with 6 ethylene oxide units to 80 ethylene oxide units and polyols are, for example, suitable as liquid carrier substances.

Of the amphiphilic organic solvents propylene glycol derivatives (PPG) are, for example, suitable such as the esters of propylene glycol and fatty acids and the ethers of PPG and fatty alcohols, such as PPG-oleyl ether and PPG oleate.

Of the lipophilic organic solvents fatty acid esters such as diisopropyl adipate, dioctyl adipate, the alkyl benzoates and dioctyl maleates can be present in a corresponding liquid carrier.

It is further possible to incorporate a fat phase into the medium of the agent according to the invention in order to influence accordingly the agent of the invention in certain applications.

The fat phase can consist of up to about 50% of the total weight of the agent. The fat phase can contain an oil or a wax or also their mixtures, or fatty acids, fatty alcohols, and fatty acid esters. The oils can be selected from animal oils, vegetable oils, mineral oils or synthetic oils or liquid silicones and especially Vaseline oils, paraffin oils, isoparaffins, polyolefines, fluorinated oils and perfluorinated oils, dimethicones, cyclomethicones, and the like. In a similar manner the waxes can be selected from known animal, fossil, vegetable, mineral or synthetic oils.

In many cases it is sensible for water to be used at least in part as liquid carrier. Within the scope of a further embodiment of the present invention the invention relates, therefore, to an agent that contains at least one compound of the general formula I and water in an amount of at least 1% by weight. For example, the fraction of water can also lie significantly above the named amount of 1% by weight. Suitable amounts of water are, for example, about 1.5% by weight to about 99.5% by weight, for example, about 5% by weight to about 98% by weight, or about 10% by weight to about 95% by weight, or about 20% by weight to about 80% by weight, or about 30% by weight to about 70% by weight, or about 40% by weight to about 60% by weight.

Within the scope of a further embodiment of the present invention an agent of the invention contains as liquid carrier water or an alcohol with a boiling point of less than 120° C. in an amount of at least 30% by weight.

Within the scope according to the present invention an agent of the invention contains in addition to a compound of the general formula I at least one surfactant. In this context this is at least one anionic surfactant or at least one cationic surfactant or a mixture of two or more of them.

Suitable surfactants are the surfactants named above within the context of the present text. In a number of applications, for example, it has been found to be advantageous if an agent according to the invention contains a quaternary ammonium compound. Such agents are applicable, for example, especially as hair conditioning agents.

Within the scope according to the present invention an agent of the present invention can be modified, for example, for application to the skin, especially as sun screen.

In this case the agent according to the invention can contain, for example, a nanopigment of a metallic oxide that is selected from the oxides of titanium, cerium, zirconium, zinc, iron, or their mixtures.

Nanopigments are understood to mean pigments whose mean size of elemental particles lies above 5 nm and below 100 nm. According to a preferred embodiment of the invention this size lies below 50 nm. The nanopigments can be coated or uncoated.

Coated pigments are pigments that have been subjected to one or more surface treatments of a chemical, electronic, mechanical-chemical and/or mechanical nature with compounds that are described in, for example, “Cosmetics & Toiletries, February 1990, volume 105, p. 53-64,” for example, amino acids, bees wax, fatty acids, fatty alcohols, anionic surface-active substances, lecithins, the sodium, potassium, zinc, iron, or aluminum salts of fatty acids, metal alkoxides (of titanium or aluminum), polyethylene, silicones, proteins (collagen, elastin), alkanolamines, silicon oxides, metal oxides, or sodium hexametaphosphate.

Silicones are, of course, known to be organosilicon polymers or oligomers of straight-chain or cyclic, branched or crosslinked structure with variable molecular weight that are prepared by polymerization and/or polycondensation of suitably functionalized silanes and which consist substantially of repeated main units, wherein the silicon atoms are bonded to each other through oxygen atoms (siloxane bonding), whereby optionally substituted hydrocarbon groups are directly bonded to the silicon atom through a carbon atom. The expression “silicones” includes also the silanes and especially the alkylsilanes necessary for their preparation.

Silicones that are suitable according to the present invention for the coating of nanopigments are preferably selected from alkylsilanes, polydialkylsiloxanes and polyalkylhydrogensiloxanes. More preferred are the silicones selected from octyltrimethylsilane, polydimethylsiloxanes, and polymethylhydrogensiloxanes.

Nanopigments of metal oxides can naturally be treated with other surface-active substances prior to treatment with silicones, especially cerium oxide, aluminum oxide, silicon oxide, alumina compounds, silicon compounds, or their mixtures.

In the case of the coated pigments these are especially titanium oxides that are coated with: silicon oxide, silicon oxide and iron oxide, silicon oxide and alumina, alumina, alumina and aluminum stearate, alumina and aluminum laurate, iron oxide and iron stearate, zinc oxide and zinc stearate, silicon oxide and alumina and, treated with a silicone, silicon oxide, alumina, aluminum stearate and, treated with a silicone, alumina and, treated with a silicone, triethanolamine, stearic acid, or sodium hexametaphosphate.

Mixtures of metal oxides can also be used, especially those of titanium dioxide and cerium dioxide, including the mixture of equal amounts by weight of titanium dioxide and cerium dioxide that are coated with silicon oxide as well as a mixture of titanium dioxide and zinc dioxide that is coated with alumina, silicon oxide, and silicone.

The nanopigments can be present in the agents according to the invention in concentrations that generally lie in the range of 0.1% by weight to 30% by weight and preferably in the range of 0.5% by weight to 10% by weight relative to the total weight of the composition.

In addition to the nanopigments the agents according to the invention can also contain conventional non-colored pigments with a normal size in the range of 100 nm to 700 nm, for example, zinc oxide, titanium dioxide “FT. HOMBITAN” with a mean size of elemental particles of 400 nm that is traded by the company SACHTLEBEN CHEMIE GmbH, or zinc oxide “NEIGE” from the company LAMBERT RIVIERE, or also colored pigments, for example, iron oxides “FDC RED 40 (37011/90119)” from the company ANSTEAD and “SICOMET BRUN ZP 3569” and “SICOVIT DAUNE 10 E 172” from the company BASF.

The cosmetic agents according to the invention can naturally also contain one or more hydrophilic or lipophilic organic sun screen filters that absorb in the UV-A and UV-B range. These filters can be selected especially from the cinnamates, salicylates, benzylidenecamphor derivates, triazine derivates, benzophenone derivates, benzotriazole derivates, ββ-diphenylacrylate derivates, dibenzoylmethane derivates, p-aminobenzoates, polymer filters, and silicone filters that are described in Patent Application No. WO 93/04665. Further examples of organic filters are described in European Patent Application No. 0 487 404. The organic filters can be present in the agents according to the invention in concentrations of from 0.1% by weight to 30% by weight relative to the total weight of the composition.

The agents according to the invention can also contain agents for tanning and/or artificial coloration of the skin (self-tanning agents), for example, dihydroxyacetone (DHA).

The agents according to the invention also contain a cosmetically acceptable aqueous medium. They exhibit a pH value that can lie in the range of from 3.5 to 11, preferably from 5.5 to 11, and more preferably from 5.5 to 8.5.

The agents according to the invention can be present in any arbitrary form suitable for topical application, especially as lotions, vesicle dispersions, simple or complex emulsions (o/w, w/o, o/w/o, w/o/w), for example, as cream or milk, aqueous or aqueous-alcoholic gels or gel-creams, in powder form, as pastes and they can optionally be configured as aerosols and in the form of foam or spray. Preferably the agents are present in the form of a gel-cream. They can be prepared by normal methods.

The agents according to the invention can be used, for example, as agents for the protection of the human epidermis or the hair against UV radiation, as sun screen agents or as make-up products.

If the cosmetic preparation according to the invention is used for the protection of the human epidermis against UV radiation or as sun screen agent it can be present as a suspension or dispersion in solvents or fat substances, in the form of nonionic vesicle dispersions or also as an emulsion, for example, as a cream or milk, ointment, gel, gel-cream, aerosol foam, or spray.

An agent of the present invention can contain, for example, at least the following components:
a) about 0.0001% by weight to about 10% by weight of a compound of the general formula I;
b) about 1% by weight to about 99% by weight water or alcohol with a boiling point of less than 120° C.;
c) about 0.1% by weight to about 90% by weight of an anionic or cationic surfactant;
d) at least 0.01% by weight of a further additive selected from the group of nonionic surfactants, zwitterionic surfactants, betaines, nonionic polymers, polysiloxanes, zwitterionic and amphoteric polymers, anionic polymers, thickening agents, structuring agents, hair conditioning compounds, perfume oils, solvents and solubilizers, active materials to improve the fiber structure of the hair, quaternary amines, amidoamines, defoamers, dyes, active antidandruff compounds, photoprotectants, substances for adjustment of the pH value, plant extracts, cholesterol, consistency regulators, fats and waxes, chelating agents, swelling and penetration materials, opacifiers, pearlescents, pigments, stabilizers, aerosol propellants, or antioxidants.

An agent according to the invention is suitable, for example, as a conditioner, deep conditioner, deep conditioner cream, deep conditioning leave-on spray, hair treatment agent with volatile materials, hair wax, shampoo, shower cream, sun screen agent, or hair & body sun screen spray. Typical representatives of the named ranges of application can have the following compositions:

Conditioner:

Cationic surfactant: about 0.1% by weight to about 5% by weight

Fatty alcohol: about 1% by weight to about 5% by weight

Compound of the General formula I: about 0.001% by weight to about 10% by weight
Further additives: about 0.01% by weight to about 10% by weight
Water: about 70% by weight to about 99% by weight
   Deep Conditioner:
Cationic surfactant: about 0.1% by weight to about 5% by weight
Fatty alcohol: about 2% by weight to about 10% by weight
Compound of the general formula I: about 0.001% by weight to about 10% by weight
Further additives: about 0.01% by weight to about 10% by weight
Water: about 70% by weight to about 99% by weight
   Hair Shampoo:
Anionic Surfactant: about 5% by weight to about 50% by weight
Compound of the general formula I: about 0.001% by weight to about 10% by weight
Further additives: about 0.01% by weight to about 10% by weight
Water: about 50% by weight to about 95% by weight
   Hair Wax:
O/W emulsifier: about 5% by weight to about 10% by weight
Compound of the general formula I: about 0.001% by weight to about 10% by weight
Further additives: about 0.01% by weight to about 10% by weight
Wax components: about 5% by weight to about 30% by weight
Water: about 50% by weight to about 80% by weight

The compounds of the invention is basically suitable for the non-therapeutic preventative, conditioning, detersive, or cosmetic treatment of skin or hair. Object of the present invention is thus also a method for the non-therapeutic preventative, conditioning, detersive, or cosmetic treatment of skin or hair in which the skin or hair is brought into contact with an agent of the invention.

The invention is illustrated more closely by examples in the following.

### EXAMPLES

Pyrimidinols and pyridinols are able to protect hair color and hair structure from damage caused by the effect of light. The protective action covers not only the achievement of a visible effect but is also measureable in a sunlight simulation investigation. The measurement values documented in the following serve only the quantification of effects already visible to the naked eye. In addition to the retention of the natural or artificially applied hair color, technical measurements shows an improved retention of the fiber structure of the hair treated with a care product containing pyrimidinols or pyridinols or a mixture of two or more of them.

The damage to hair structure necessarily associated with radiation can be significantly reduced by use of a product containing pyrimidinols or pyridinols or a mixture of two or more of them. Thus the peak temperatures measured after treatment with a pre-sun product of the products containing pyrimidinols or pyridinols or a mixture of two or more of them lay in all cases significantly higher than that hair treated with conventional products. The peak temperature in the HPDSC measurement represents a measure of the support against denaturation of the +α-helical regions of the intermediate filaments by keratin-associated proteins. The more the hair is damaged the lower the denaturation temperature in the HPDSC.

### EXAMPLES

### Example 1

### Conditioner

| | | |
|---|---|---|
| 4.3 | g | cetearyl alcohol (Lanette ® O) |
| 0.4 | g | cetyl lactate |
| 0.5 | g | Vaseline ® (petroleum jelly) |
| 1.2 | g | cetyltrimethylammonium chloride |
| 0.45 | g | polyvinylpyrrolidone |
| 0.02 | g | 2-dimethylamino-4,6-dimethyl-5-pyrimidinol |
| balance to 100 | g | water |

### Example 2

### Deep Conditioner

| | | |
|---|---|---|
| 5.5 | g | cetearyl alcohol (Lanette ® O) |
| 1.2 | g | Vaseline ® (petroleum jelly) |
| 1.0 | g | liquid paraffin |
| 0.5 | g | dimethylpolysiloxane (Belsil ® DM 500) |
| 0.3 | g | lanolin alcohol |
| 0.2 | g | lanolin |
| 1.2 | g | cetyltrimethylammonium chloride |
| 0.3 | g | citric acid |
| 0.4 | g | perfume |
| 0.02 | g | 2-dimethylamino-4,6-dimethyl-5-pyrimidinol |
| balance to 100 | g | water |

### Example 3

### Deep Conditioning Cream

| | | |
|---|---|---|
| 6 | g | cetearyl alcohol |
| 1.7 | g | glycerol |
| 1 | g | cetyltrimethylammonium chloride |
| 1 | g | vegetable oil |
| 0.5 | g | panthenol |
| 0.5 | g | silicon oil (dimethylpolysiloxane) |
| 0.2 | g | perfume |
| 0.02 | g | 2-dimethylamino-4,6-dimethyl-5-pyrimidinol |
| balance to 100 | g | water |

### Example 4

### Deep Conditioner Leave-On Spray

| | | |
|---|---|---|
| 0.5 | g | cetyl alcohol |
| 0.3 | g) | glycerol |
| 0.25 | g | cetyltrimethylammonium chloride |
| 0.2 | g | styrene/vinylpyrrolidone copolymer |
| 0.2 | g | panthenol |
| 0.01 | g | 2-dimethylamino-4,6-dimethyl-5-pyrimidinol |
| 0.2 | g | perfume |
| 4.8 | g | ethanol |
| balance to 100 | g | water |

### Example 5

### Hair Treatment Agent With Highly Volatile Material

| | | |
|---|---|---|
| 10 | g | propylene glycol |
| 2 | g | cetearyl alcohol (Lanette ® O) |
| 2 | g | cyclomethicone (Dow Corning 345 Fluid) |
| 0.4 | g | perfume |
| 0.2 | g | PHB methyl ester |
| 0.1 | g | PHB propyl ester |
| 0.002 | g | 2-dimethylamino-4,6-dimethyl-5-pyrimidinol |
| balance to 100 | g | water |

### Example 6

### Hair Wax

| | | |
|---|---|---|
| 10 | g | triceteareth-4 phosphate |
| 6 | g | PEG-200 hydrogenated glyceryl palmate |
| 18 | g | caprylic/capric triglyceride |
| 0.5 | g | perfume |
| 0.5 | g | preservative |
| 0.01 | g | 2-dimethylamino-4,6-dimethyl-5-pyrimidinol |
| balance to 100 | g | water |

### Example 7

### Shampoo

| | | |
|---|---|---|
| 30 | g | sodium lauryl ether sulfate |
| 8 | g | cocamidopropyl betaine |
| 3 | g | glycol distearate |
| 0.5 | g | perfume |
| 0.35 | g | sodium benzoate |
| 0.15 | g | sodium formate |
| 0.2 | g | sodium chloride |
| 0.2 | g | 2-dimethylamino-4,6-dimethyl-5-pyrimidinol |
| balance to 100 | g | water |

### Example 8

### Shower Cream

| | | |
|---|---|---|
| 12 | g | 70% sodium lauryl ether sulfate |
| 0.7 | g | preservative |
| 0.4 | g | perfume |
| 7.5 | g | opacifier (20 percent by weight PEG-3 distearate |
| | | and 18 percent by weight sodium lauryl ether |
| | | sulfate in water) |
| 0.3 | g | polyquaternium-10 (cationic cellulose) |
| 0.4 | g | PEG-7 glyceryl cocoate |
| 0.3 | g | D-panthenol |
| 0.2 | g | 2-dimethylamino-4,6-dimethyl-5-pyrimidinol |
| 5 | g | cocamidopropyl betaine, 30% in water |
| 1 | g | NaCl |
| balance to 100 | g | water |

### Example 9

### Sun Screen

| | INCI/EU | % by weight |
|---|---|---|
| oil phase: | | |
| Parsol 1789 | butylmethoxydibenzoylmethane | 0.30 |
| Neo Heliopan AV/OA | octyl methoxycinnamate | 10.00 |
| Hostaphat KL 340 N | trilaureth-4 phosphate | 0.60 |
| Hostacerin DGI | polyglyceryl-2 | 0.70 |
| | sesquiisostearate | |
| phenoxetol | phenoxyethanol | 1.00 |
| Cetiol 868 | octyl stearate | 5.00 |
| Primol 352 | mineral oil | 5.00 |
| aqueous phase: | | |
| Carbopol 2984 | carbomer | 0.30 |
| sodium hydroxide | sodium hydroxide | 0.06 |
| glycerol 86% | glycerol | 5.00 |
| 2-dimethylamino-4,6- | | 0.01 |
| dimethyl-5-pyrimidinol | | |
| water, completely | water | balance |
| desalinated | | to 100 |

### Example 10

### Sun Screen

| | INCI/EU | % by weight |
|---|---|---|
| oil phase: | | |
| Parsol 1789 | butylmethoxydibenzoylmethane | 0.30 |
| Neo Heliopan AV/OA | octyl methoxycinnamate | 10.00 |
| Hostaphat KL 340 N | trilaureth-4 phosphate | 0.60 |
| Hostacerin DGI | polyglyceryl-2 | 0.70 |
| | sesquiisostearate | |
| phenoxetol | phenoxyethanol | 1.00 |
| Cetiol 868 | octyl stearate | 9.00 |
| Abil Wax 9801D | cetyl dimethicone | 1.00 |
| aqueous phase: | | |
| Carbopol 2984 | carbomer | 0.30 |
| sodium hydroxide | sodium hydroxide | 0.06 |
| glycerol 86% | glycerol | 5.00 |
| 2-dimethylamino-4,6- | | 0.01 |
| dimethyl-5-pyrimidinol | | |
| water, completely | water | balance |
| desalinated | | to 100 |

### Example 11

### Sun Screen

| | INCI/EU | % by weight |
|---|---|---|
| lipid phase: | | |
| Parsol 1789 | butyl | 1.50 |
| | methoxydibenzoylmethane | |
| PHB methyl ester | methylparaben | 0.20 |
| Neo Heliopan AV/OA | octyl methoxycinnamate | 10.00 |
| Neo Heliopan Type 303 | octocrylene | 10.00 |
| Finsolv TN | C12-15 alkyl benzoates | 2.50 |
| Eutanol G | octyldodecanol | 10.00 |
| Antaron V 216 | PVP/hexadecene copolymer | 2.00 |
| vitamin E acetate | tocopheryl acetate | 0.50 |
| perfume | perfume | 0.30 |
| Abil Wax 9801D | cetyl dimethicone | 0.50 |
| aqueous phase: | | |
| Carbopol 1382 | acrylates/C10-30 alkyl acrylate | 0.45 |
| | crosspolymer | |
| Colorona Oriental Beige | MICA (and) CI 77891 (and) | 0.05 |
| 17237 | CI 77491 | |
| glycerol 86% | glycerol | 5.00 |
| Edeta BD | disodium EDTA | 0.10 |
| sodium hydroxide | sodium hydroxide | 0.18 |
| D-panthenol | panthenol | 0.50 |
| 2-dimethylamino-4,6- | | 0.01 |
| dimethyl-5-pyrimidinol | | |
| water, completely | water | balance |
| desalinated | | to 100 |
| Dekaben LMB | iodopropynylbutyl carbamate | 0.50 |

### Example 12

### Hair & Body Sunscreen Spray

| lipid phase: | INCI/EU | % by weight |
|---|---|---|
| Parsol 1789 | butylmethoxydibenzoylmethane | 0.50 |
| Neo Heliopan AV/OA | octyl methoxycinnamate | 2.00 |
| Finsolv TN | C12-15 alkyl benzoates | 2.50 |
| Eutanol G | octyldodecanol | 10.00 |
| JAGUAR c-13-S | guar hydroxypropyltrimonium | 0.50 |
| | chloride | |
| vitamin E acetate | tocopheryl acetate | 0.50 |
| perfume | perfume | 0.30 |
| D-panthenol | panthenol | 0.50 |
| 2-dimethylamino-4,6- | | 0.02 |
| dimethyl-5-pyrimidinol | | |
| ethanol aqueous | alcohol denat. | 60.0 |
| water, completely | water | balance |
| desalinated | | to 100 |

Determination of Tensile Strength:
Hair material:

Fine machine tresses, Kerling company, color 8/0, length below the binding 16 cm, width 3.5 cm, weight without binding 0.8 g, cysteic acid value: 21.1 μmol/g hair

Hair Pretreatment:

The hair is washed with in each case 0.2 mL standard shampoo (10% Na-LES, 4% NaCl) per strand for 1 minUTE and rinsed for 1 minUTE under tap water (35° C.). The strands are dried at least overnight in a conditioning room at 20° C. and 65% relative humidity.

Application of the Test Samples:

Application of the test samples: The dry locks of hair are irradiated with a Xenotest Alpha instrument from the Atlas company (with Xenochrome filter 270) at 85% relative humidity and 35° C. for 16 hours with 600 W/m². They are then placed in warm tap water for 15 minutes at 35° C., patted dry so that they have a residual moisture content of 50% by weight. For example 1 the locks of hair are not treated with a product, for examples 2 and 3 the locks are in each case treated with 0.2 mL product (example 2: deep conditioner without a compound of general formula I, example 3: deep conditioner with a compound of general formula I) per lock and are rubbed in evenly for one minute. The product is allowed to act for 10 minutes and is then rinsed out under running tap water at 35° C. for one minute. Next the locks are shampooed with 7.5 mL standard shampoo per lock for 1 minute and rinsed for 1 minute with water (35° C.) and dried in the conditioning room (20° C./65% relative humidity).

Measurement of Tensile Strength:

Tensile strength was determined as follows:

Three hair strands are prepared for each example. For tensile strength measurement single hairs are taken from each dry lock and metal sleeves are applied terminally on both sides (crimped). A total of 25 single hairs per sample are used to measure tensile strength.

The crimped hairs are stored overnight in the conditioning room after which the individual hair diameters are determined by a computer-controlled laser micrometer (Zimmer company, Roβdorf). Next the stress-strain diagram is measured for each individual hair with a tensile tester (MTT 160/600 Series Miniature Tensile Tester, Serial No. 600.95.05.001, Fa. DIA-STRON Ltd., England). Breaking is carried out on wet hair.

For evaluation and representation in diagrams the tensile strength of the individual hairs, which varies greatly from one another owing to the different hair diameters, are first standardized to a mean diameter or mean hair cross-section (standardization of the 25 individual values of a lock with consideration of the respective hair diameter to a hair diameter of 0.08 mm (mean diameter) by linear regression).

A new evaluation method is used for the hair protection investigation: with consideration of the respective hair diameter the tensile strength for a hair diameter of 0.08 mm was calculated and converted into unit peak load (N).

Results:

Example 1 (without treatment with deep conditioner):
   Tensile strength=0.741 N
Example 2 (deep conditioner without a compound of the general formula I):
   Tensile strength=0.786 N
Example 3 (deep conditioner with a compound of the general formula I):
   Tensile strength=0.812 N

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is

1. An agent for the protective, conditioning, detersive or cosmetic treatment of skin or hair comprising at least one liquid carrier, one surfactant and one compound of the general formula I 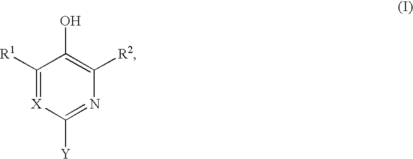
wherein X stands for N or C, R¹ stands for a linear or branched, saturated or unsaturated C₁₋₂₄-alkyl residue, a phenyl residue, a C₇₋₂₈-phenylalkyl residue, whereby the phenylalkyl residue can be substituted on the phenyl ring by fluorine, chlorine, C₁₋₆-alkyl, C₁₋₆-alkoxy. CF₃, furfuryl or thienyl, R² stands for hydrogen, C₁₋₂₄-alkyl, phenyl, C₇₋₂₈-phenylalkyl, whereby the phenylalkyl residue can be substituted on the phenyl ring by fluorine, chlorine, C₁₋₆-alkyl, C₁₋₆-alkoxy. CF₃, furfuryl or thienyl, and Y stands for NH₂, NHR³ or NR³R⁴, wherein R³ und R⁴, independently of one another, stand for a linear or branched, saturated or unsaturated C₁₋₁₂-alkyl group or a linear or branched, saturated or unsaturated C₁₋₁₂-hydroxyalkyl group, or R³ and R⁴ together form a ring so that Y stands for an imidazolyl group or a quinolinyl group, or a morpholinyl group or a pyrazolyl group or a piperidinyl group or Y stands for a linear or branched, saturated or unsaturated C₁₋₁₂-alkoxy group, a linear or branched, saturated or unsaturated C₁₋₁₂-hydroxyalkoxy group, a linear or branched, saturated or unsaturated C₁₋₁₂-alkylcarboxylic acid group, a linear or branched, saturated or unsaturated C₁₋₂₄-alkylcarbamoyl group, a linear or branched, saturated or unsaturated C₁₋₁₂-alkylsulfonic acid group, a linear or branched, saturated or unsaturated C₁₋₂₄-alkylsulfonic acid ester group or a linear or branched, saturated or unsaturated C₁₋₂₄-alkylsulfonamide group or wherein R³ together with X form an anelated ring with 5 atoms or 6 atoms, or a mixture of two or more of them, whereby the content of the agent of compounds of the general formula I is less than 10% by weight.

2. An agent according to claim 1, comprising, as liquid carrier water, or an alcohol with a boiling point of less than 120° C. in an amount of at least 30% by weight.

3. An agent according to claim 1, comprising at least one anionic surfactant or at least one cationic surfactant or a mixture of two or more of them.

4. An agent according to claim 1, comprising a quaternary ammonium compound.

5. An agent according to claim 1, comprising:
a) about 0.0001% by weight to about 10% by weight of a compound of general formula I;
b) about 1% by weight to about 99% by weight water or alcohol with a boiling point of less than 120° C.;
c) about 0.1% by weight to about 90% by weight of an anionic or cationic surfactant;
d) at least 0.01% by weight of a further additive selected from the group consisting of non-ionic surfactants, zwitterionic surfactants, betaines, nonionic polymers, polysiloxanes, zwitterionic and amphoteric polymers, anionic polymers, thickening agents, structuring agents, hair conditioning compounds, perfume oils, solvents and solubilizers, active compounds that improve the fiber structure of the hair, quaternary amines, amidoamines, defoamers, dyes, active antidandruff compounds, photoprotective agents, substances to adjust the pH value, plant extracts, cholesterol, consistency regulators, fats and waxes, chelating agents, swelling and penetration materials, opacifiers, pearlescents, pigments, stablizers, aerosol propellants, or antioxidants.

6. A method for the non-therapeutic, protective, conditioning, detersive or cosmetic treatment of skin or hair by which the skin or hair is brought into contact with an agent according to claim 1.
